(19) United States
(12) Patent Application Publication
(10) Pub. No.: US 2026/0355111 A1
(43) Pub. Date: Nov. 12, 2026

(54) EXCIPIENT-FREE LYOPHILIZATION OF POLYMER LUNG SURFACTANTS

(71) Applicant: Purdue Research Foundation, West Lafayette, IN (US)

(72) Inventors: You-Yeon Won, West Lafayette, IN (US); Sungwan Park, West Lafayette, IN (US); Seyoung Kim, West Lafayette, IN (US); Daniel James Fesenmeier, Lafayette, IN (US)

(21) Appl. No.: 19/118,746

(22) PCT Filed: Oct. 4, 2023
(86) PCT No.: PCT/US2023/075931

§ 371 Date: Apr. 4, 2025

Related U.S. Application Data
(60) Provisional application No. 63/378,470, filed on Oct. 5, 2022.

(51) Int. Cl. A61K 9/107; A61K 9/19; A61K 31/765; A61K 31/785; C08F 283/06

(52) U.S. Cl. CPC A61K 9/1075; A61K 9/19; A61K 31/765; A61K 31/785; C08F 283/06

(57) ABSTRACT

The present disclosure relates to a protocol for the formulation and lyophilization of hydroxyl-terminated poly(ethylene glycol) (PEG) based block copolymer micelles without the use of excipients (cryo-protectants). The micelles are formulated using the equilibrium nanoprecipitation (ENP) method, which allows for control over the aggregation number of the micelles and thus the grafting density of the PEG micelle corona chains. By using the ENP method to produce micelles with high dimensionless PEG grafting densities (> about 7), the micelles are stabilized against aggregation during lyophilization due to the shielding of the micelle core domains by the dense PEG corona chains. By preventing aggregation during the lyophilization without using excipients, the original desired surface mechanical properties of the micelles are preserved after reconstitution. Therefore, the protocol described herein can be applied to effectively storing PEG-based block copolymer micelles without using excipients while still preserving their original surface mechanical properties.

### CROSS-REFERENCE TO RELATED APPLICATIONS

This application claims the benefit of U.S. Provisional Appl. Ser. No. 63/378,470, filed Oct. 5, 2022, which is incorporated by reference as if fully set forth herein.

### GOVERNMENT SUPPORT CLAUSE

This invention was made with government support from the National Science Foundation (NSF) under grant CBET-2211843. The government has certain rights in the invention.

### BACKGROUND

Acute respiratory distress syndrome (ARDS) has a mortality rate of 25 to 40%. ARDS annually affects approximately 200,000 patients in the United States and 3 million patients globally (Matthay & Zemans, 2011; Reamaroon et al., 2020; Sasannejad et al., 2019). ARDS has been identified as the leading cause of SARS-CoV-2-related deaths during the recent COVID-19 outbreak. ARDS is initiated by various types of lung injuries, such as inhalation of pathogens, pneumonia, and sepsis, and is exacerbated through a cascade of events that deactivate lung surfactant, which plays a crucial role in the breathing process. One pathology in the development of ARDS is the deactivation of natural lung surfactant, a substance that regulates the alveolar air-water interfacial tension, by serum proteins and phospholipases that are present in the lungs during injury and inflammation. The result of lung surfactant deactivation is an increased air-water surface tension, destabilization of the alveolar network, and impaired oxygenation. In the early stages of ARDS, inflammation leads to pulmonary edema by increasing capillary permeability, causing surface-active plasma proteins like albumin and hemoglobin to enter the alveolar spaces. Additionally, elevated levels of phospholipase A2 (PLA₂) in ARDS lungs result in the accumulation of single-chain lysolipids and fatty acids by hydrolyzing double-chain phospholipids. This deactivation mechanism undermines the efficacy of the current standard therapy, which involves replacing damaged lung surfactant with animal-extracted exogenous surfactant, as it is also susceptible to deactivation. Consequently, there is an urgent need to discover and develop an artificial lung surfactant that is resistant to the deactivation mechanisms of ARDS. Further, there is currently no surfactant replacement therapy (SRT) that has been shown to be effective in treating ARDS in adults. There is therefore in impetus for exploring the use of a novel fully synthetic polymer lung surfactant (PLS) comprised of amphiphilic block copolymer micelles for treatment of ARDS and other similar symptoms including acute lung injury (ALI) and neonatal respiratory distress syndrome (NRDS).

### SUMMARY

The disclosure provides, among other things, a method of lyophilizing PLS (e.g., PS-PEG micelle PLS) without excipients for long-term storage and ease of handling and transportation without compromising its surface tension-lowering property post reconstitution in an aqueous medium. Embodiments provide, for example, processes for preparing PS-PEG micelles having a PEG grafting density (σ_{PEG}) of about 5 or greater (i.e., σ_{PEG}>5 for 5 kDa PEG, but this lower limit for σ_{PEG} can be smaller for higher molecular weight PEG). As used herein, σ_{PEG} is defined as the ratio πR_{g}²/α, where R_{g} is the radius of gyration of the PEG block in the unperturbed (free) hydrated state, and α is the micelle core surface per block copolymer chain.

In one example, the methods described herein comprise the steps of: (a) forming monodisperse block copolymer micelles via equilibrium nanoprecipitation (ENP) initially in a water-organic cosolvent mixture at a water volume fraction (φ_{w,ENP}) between about 10% and about 80% (e.g., from about 20% to about 70%, about 10% to about 60%, about 20% to about 30%, about 10% to about 30%, about 30% to about 60%, about 10% to about 50%, about 10% to about 50% or about 20% to about 50%); (b) conducting a single-step dialysis against a water reservoir to remove the organic solvent from the block copolymer micelle solution; (c) freezing the aqueous micelle solution (for example at −60 °C); (d) sublimating the crystalized water from the micelle solution (e.g., at −30 °C and 70 mTorr); and (e) desorbing and vaporizing the residual supercooled water bound to the polymer micelles (e.g., at 25 °C and 70 mTorr).

In one example, the reconstitution process after the lyophilization comprises: (a) dissolving the lyophilized block copolymer micelles in an aqueous medium; (b) manually shaking or vortexing the micelle solution (e.g., for three minutes); and (c) further sonicating the micelle solution for three minutes to ensure that the polymer micelles are fully dissolved and the micelle solution becomes transparent.

An alternative to lyophilization is spray drying which can also convert a block copolymer micelle PLS formulation from a liquid to a powder. In spray drying, a liquid formulation is rapidly dried with a hot gas, whereas in lyophilization (freeze drying), water is removed below its freezing temperature under high vacuum.

### DESCRIPTION OF THE FIGURES

To facilitate a better understanding of the present disclosure, reference will be made to the embodiments demonstrated in drawings.

FIG. 1 is a plot of surface pressure-area isotherms of PS-PEG-OH micelles spread from 10% w/v excipient-containing formulations on pure water surfaces. The micelles were prepared using the equilibrium nanoprecipitation (ENP) method in a mixed solvent of water and acetone, with a water volume fraction of φw,ENP=0.6. The control measurement (black line) represents micelles without any excipients. Data points were collected at intervals of 0.3 cm² (solid lines), with only a few data points shown (symbols) for clarity.

FIGS. 2A-2D are plots showing shelf temperature and heat flux profiles during initial freezing stages for lyophilization Method A and Method B, respectively. The negative heat flux values indicate exothermic processes. The shaded regions represent the time intervals between the onset of nucleation and the minimum heat flux.

FIG. 3A-3H are transmission electron microscope (TEM) images of pristine PS-PEG-OH micelles formulated at φ_{w,ENP}=0.2, 0.3, 0.5, and 0.6, respectively (FIGS. 3A-3D), and reconstituted micelles thereof after lyophilization (Method A) (FIGS. 3E-3H).

FIGS. 4A-4D are plots of pressure-area (Π-A) isotherms of pristine, reconstituted, and solid-spread PS-PEG-OH micelle monolayers. Micelles were formulated at φ_{w,ENP}=0.2 (FIG. 4A), 0.3 (FIG. 4B), 0.5 (FIG. 4C), and 0.6 (FIG. 4D). The inset of FIG. 4A shows a representative overlap between the rescaled curve of the pristine multiplied by a factor (“surface availability (SA)”) of 0.36 and the curve of the reconstituted micelles. The abscissa (area in cm²) is presented on a logarithmic scale.

FIGS. 5A-5H are TEM images of pristine PS-PEG-OCH₃ micelles formulated at φ_{w,ENP}=0.1, 0.2, 0.3, and 0.5, respectively (FIGS. 5A-5D), and reconstituted micelles thereof after lyophilization (Method A) (FIGS. 5E-5H).

FIGS. 6A-6D are plots of Π-A isotherms of pristine, reconstituted, and solid-spread PS-PEG-OCH₃ micelle monolayers. Micelles were formulated at φ_{w,ENP}=0.1 (FIG. 6A), 0.2 (FIG. 6B), 0.3 (FIG. 6C), and 0.5 (FIG. 6D).

FIGS. 7A-7H are TEM images of pristine PtBMA-PEG-OCH₃ micelles formulated at φ_{w,ENP}=0.1, 0.2, 0.3, and 0.5, respectively (FIGS. 7A-7D), and reconstituted micelles thereof after lyophilization (Method A) (FIGS. 7E-7H).

FIGS. 8A-8D are plots of Π-A isotherms of pristine, reconstituted, and solid-spread PtBMA-PEG-OCH₃ micelle monolayers. Micelles were formulated at φ_{w,ENP}=0.1 (FIG. 8A), 0.2 (FIG. 8B), 0.3 (FIG. 8C), and 0.5 (FIG. 8D).

FIGS. 9A-9F are DSC thermograms of PS-PEG-OH (FIG. 9A), PS-PEG-OCH₃ (FIG. 9B), and PtBMA-PEG-OCH₃ (FIG. 9C) micelle suspensions at concentrations ranging from 1 to 10 wt %. The micelles were formulated at different φ_{w,ENP} values as indicated in the legends. A cooling-and-heating cycle was performed at a rate of 5 °C/min. The insets provide enlarged views at the eutectic point. Correlations between the fraction of hydrated PEG (evaluated from the eutectic melting enthalpy in the DSC study) (FIGS. 9D-9F) and the size increase factor (SIF) after lyophilization (FIG. 9D), the nondimensional PEG grafting density of micelles (FIG. 9E), and the surface availability (SA) (FIG. 9F). Open symbols represent samples with low fidelity due to low polymer concentration in the analytes (w_{p,analyte}<0.02).

FIGS. 10A-10C is a schematic representation of an aqueous micelle system during lyophilization stages (FIG. 10A). (i) Freezing stage: Bulk ice crystallizes, leading to the condensation and steric compression of micelles (depicted with arrows as “freezing stress”). (ii) Primary drying stage: Bulk ice sublimates, leaving behind “bound” water present as an amorphous PEG/water mixture (“amorphous layer”). (iii) Secondary drying stage: Bound water evaporates. In aqueous media, PEG segments in the PS-PEG-OH micelle coat the hydrophobic PS surface to reduce surface energy (FIG. 10B). After forming a dehydrated PEG film, the remaining PEG segments can interact with water. The fraction of PEG that interacts with water increases with higher grafting density (σ_{PEG}), enhancing the dissipating effect of the amorphous layer against freezing stress. PS-PEG-OCH₃ and PtBMA-PEG-OCH₃ micelles exhibit similar hydrated PEG fractions compared to PS-PEG-OH micelles (FIG. 10C). However, the methoxy end groups promote particle aggregation through bridging interaction, counteracting the dissipating effect of the amorphous layer.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Micelles derived from fully synthetic block copolymers, such as poly(styrene)-b-poly(ethylene glycol) (PS-PEG), are promising candidates for SRT applications (H. C. Kim et al., 2018). Block copolymer micelle-based PLS formulations prepared via the “equilibrium nanoprecipitation” (ENP) method exhibit narrow size distribution (“monodisperse”) characteristics (Won et al., 2021) and also an exceptional surface-tension lowering functionality under high compression. Unlike lipid/protein-based lung surfactant formulations, the surface-tension lowering functionality of PLS is even preserved in the presence of surface-active serum proteins such as albumin. Additionally, in vivo studies using mouse models of lung injury suggest that PLS is more efficacious than animal-derived lung surfactants without producing any toxicological effects. PLS has favorable pharmacokinetic characteristics with an alveolar clearance half-life of about 16 days following pharyngeal administration of a therapeutic dose in mice (S. Kim et al., 2022).

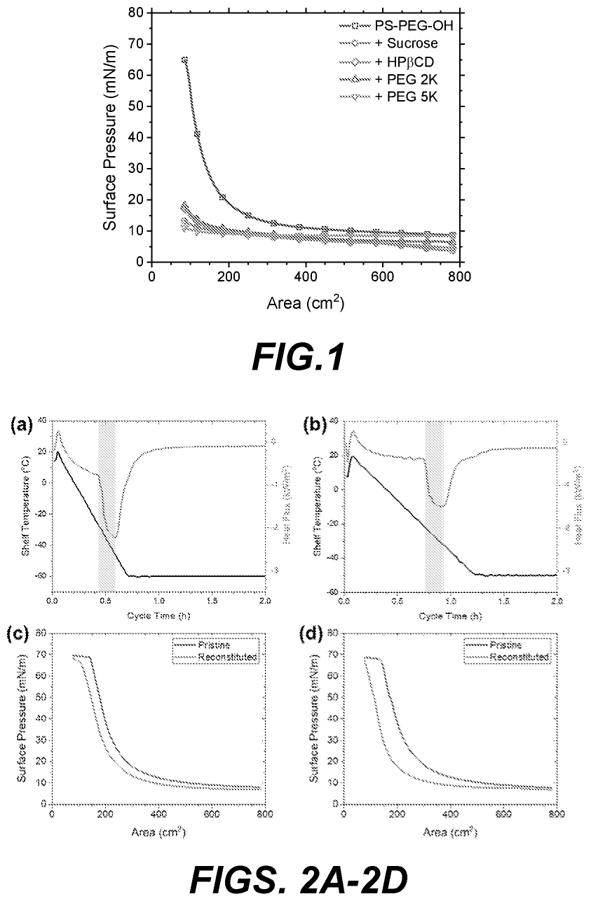

Lyophilization (freeze drying) is necessary to store PLS in the dried state. Lyophilization dramatically increases the shelf life of PLS and even enables room temperature storage of PLS. Moreover, with lyophilization, it becomes feasible to transport PLS over long distances without destabilization of the formulation. Block copolymer micelles are in general vulnerable to aggregation when stored for a long time in aqueous solution at ambient temperature (Mortensen, 1998). For lyophilization of a polymer micelle nanoparticle formulation, an excipient (alternatively called cryoprotectant or lyoprotectant) should be added to the solution prior to lyophilization in order to prevent agglomeration of the nanoparticles. The excipient molecules form a glassy rigid inert matrix which separates the nanoparticles during the freezing step; this intervening matrix prevents nanoparticles (polymer micelles) from aggregation and also provides protection of the micelles from mechanical stresses induced by ice crystallization and water sublimation during lyophilization (Chang et al., 2005; Degobert & Aydin, 2021). However, as shown in FIG. 1, the surface pressure-area isotherm measurements on PS-PEG micelle PLS in the presence of common cyroprotectants (10% w/v) show that cryoprotectants significantly compromise the surface-tension lowering functionality of PLS. Note in the figure data are plotted in terms of the surface pressure which is defined as the surface tension of the clean air-water interface (=72.5 mN/m at room temperature) minus the surface tension of the air-water interface coated with, in that case, PLS. While PLS with no added excipient shows a sharp increase in surface pressure (a sharp decrease in surface tension) under high compression, PLS in the presence of excipients becomes incapable of producing high surface pressure (low surface tension) even at maximum compression. Considering that the surface tension-reducing capability of PLS is an important attribute for use as an SRT, and added excipients compromise this capability of PLS, excipient-free lyophilization is highly beneficial for PLS.

In one approach to lyophilize PEG-based graft copolymer micelles without using excipients, Logie et al. synthesized graft copolymers containing 10-kDa PEG blocks as side chains with varying numbers of PEG side chains per copolymer molecule to control the PEG corona chain grafting density for the graft copolymer micelles (Logie et al., 2014). They found that increasing the PEG side chain number per molecule increased the stability of the micelles and prevented the aggregation of the micelles during the lyophilization process. The PEG graft densities described in Logie range from 1.5 to 6 PEGs per backbone, which would be considered PEG-based graft copolymers having medium to high PEG content. However, in this approach, graft copolymers with a sufficient number of PEG side chains must be synthesized by a multi-step “graft to” method involving the synthesis of a backbone polymer containing reactive groups and chemical conjugation of premade 10 kDa PEG chains to the reactive groups, which is not a simple process (Essa et al., 2011; Logie et al., 2014). Additionally, the surface mechanical properties of these graft copolymer micelles have not been studied; Logie et al. did not develop these graft copolymer micelles for PLS applications. In the approach described herein, it is not necessary to synthesize a copolymer containing a high PEG content in order to achieve polymer micelles having a high PEG grafting density. In the approach described herein, the ENP method is used to control the PEG grafting density of the block copolymer micelles; at high water volume fractions (also referred to herein as “volume fraction of water” and “φ_{w,ENP}”) in the initial water-organic cosolvent mixture, polymer micelles with large aggregation numbers and thus with high PEG grafting densities can be produced without having to synthesize block copolymers with high PEG contents. The present disclosure presents a protocol of formulating PEG-based block copolymer micelles with varying PEG grafting densities, optimal conditions for excipient-free lyophilization of block copolymer micelles, and the surface mechanical properties of block copolymer micelles before and after lyophilization.

The instant disclosure generally relates to compositions comprising dried amphiphilic block copolymer (BCP) micelles having a mean hydrodynamic diameter in the range of from about 5 nm to 1,000 nm (e.g., about 5 nm to about 500 nm, about 10 nm to about 50 nm, about 10 nm to about 200 nm, about 20 nm to about 100 nm, about 20 nm to about 180 nm, about 50 nm to about 120 nm, about 5 nm to about 50 nm, or about 50 nm to about 200 nm) in the original hydrated state, the micelles comprising substantially no excipients. The amphiphilic BCP micelles in the original hydrated state can also have an apparent core diameter (D_{c}) determined based on dry-state TEM images of from about 5 nm to about 50 nm (e.g., from about 5 nm to about 20 nm, about 10 nm to about 40 nm, about 10 nm to about 25 nm, about 15 nm to about 30 nm, about 15 nm to about 30 nm, about 10 nm to about 30 nm, or about 14 nm to about 24 nm); a micelle size polydispersity index (PDI) of from about 0.020 to about 0.500 (e.g., 0.030 to about 0.300, about 0.080 to about 0.200 to about 0.400, about 0.150 to about 0.350, about 0.100 to about 0.130 or about 0.02 to about 0.120); and/or a water volume fraction (φ_{w,ENP}) between about 10% and about 80% (e.g., from about 20% to about 70%, about 10% to about 60%, about 20% to about 30%, about 10% to about 30%, about 30% to about 60%, about 10% to about 50%, about 10% to about 50% or about 20% to about 50%). And when such compositions are reconstituted following lyophilization, the mean hydrodynamic diameter of the reconstituted micelles is in the range of from about 5 nm to 1,000 nm (e.g., about 5 nm to about 500 nm, about 100 to about 250 nm, about 100 nm to about 300 nm, about 50 nm to about 300 nm, about 50 nm to about 130 nm, about 60 nm to about 120 nm, or about 100 nm to about 160 nm); a polydispersity index (PDI) of from about 0.020 to about 0.500 (e.g., about 0.100 to about 0.300, about 0.100 to about 0.200, about 0.05 to about 0.150, or about 0.125 to about 0.200); a size increase factor of from about 1.00 to about 8.00 (e.g., about 1.00 to about 6.00, about 1.00 to about 4.00, about 2.00 to about 6.00, about 4.00 to about 8.00, about 2.50 to about 5.50, about 1.80 to about 2.00, or about 1.80 to about 4.50), which is defined as the mean hydrodynamic diameter (also referred to herein as the “z-average hydrodynamic diameter (<Dₕ>)”) of the micelles before lyophilization divided by the mean hydrodynamic diameter of the micelles after lyophilization/reconstitution; and/or a surface availability of from about 0.01 to about 5 (e.g., about 0.1 to about 1, about 0.1 to about 0.5, about 0.2 to about 2, about 0.05 to about 0.3, about 0.1 to about 1, about 0.1 to about 0.4, about 0.3 to about 0.9, or about 0.4 to about 0.8). The Surface Availability (SA) is calculated as the multiplicative factor required to superimpose the surface pressure-area isotherm of the pristine micelle with that of the reconstituted micelle. For example, an SA value of 0.36 at φ_{w,ENP}=0.2 indicates that only 36% of surface-active micelles from the reconstituted formulation are available. As the micelles become more densely grafted with PEG, the Π-A isotherms of the reconstituted formulations approach those of the pristine micelles, resulting in an increase in SA close to unity (see Table 2 herein). This demonstrates the feasibility of excipient-free lyophilization when the pharmaceutical formulation consists of densely PEG-grafted micelles.

The instant disclosure also generally relates to a method of preparing compositions comprising amphiphilic BCP micelle polymer lung surfactant (PLS) in aqueous solution, using the equilibration nanoprecipitation (ENP) process, the method comprising:
(a) dissolving an amphiphilic BCP in a water-organic cosolvent mixture; and
(b) conducting a single-step dialysis against an aqueous reservoir to form kinetically frozen BCP micelles dispersed in an aqueous medium.

As used herein, the term “equilibration nanoprecipitation” or “ENP,” generally refers to a method comprising the steps of: (1) forming and equilibrating BCP micelles in a water/organic cosolvent mixture at water compositions between about 10% and about 90% (w/w), and (2) dialyzing the BCP solution against an aqueous reservoir to freeze the micelle structure and to remove or lower the non-aqueous solvent content.

By forming BCP micelles in a uniformly mixed cosolvent environment and allowing time for equilibration and afterward quickly removing the organic cosolvent past the critical water concentration (CWC), kinetically frozen micelles with narrow size distribution characteristics can be produced. The pre-equilibration procedure followed by a single-step dialysis helps achieve a better micelle size monodispersity than previous literature procedures. Previous stepwise dialysis methods for producing monodisperse BCP micelles start with a block copolymer dissolved in an organic cosolvent and involve multiple dialysis steps using water/organic cosolvent reservoirs with gradually increasing water concentration in the reservoir at each dialysis step. To the contrary, this disclosure uses an aqueous reservoir for a single-step dialysis after equilibration of the micelles in a water/organic cosolvent mixture. A single-step dialysis against a water reservoir makes the pre-formed micelle solution rapidly cross the CWC, and as a result, the micelles become kinetically frozen from their original equilibrium state in the water/cosolvent mixture. Because the size and size distribution properties of the micelles affect their therapeutic performance, the capability of the ENP method to produce monodisperse BCP micelles is a desirable feature for the PLS formulation process.

As used herein, the term “amphiphilic block copolymer” includes amphiphilic block copolymers comprising a hydrophilic (water-soluble) block comprising an ethylene glycol (or ethylene oxide) monomer unit (such as poly(ethylene glycol) (PEG) or poly(ethylene oxide) (PEO) block) and a hydrophobic (water-insoluble) block comprising a monomer unit selected from a group consisting of lactic acid or lactide (LA), glycolic acid or glycolide (GL), caprolactone (CL), styrene(S), methacrylate (MA), acrylate (AC), butadiene (BD), and isoprene (IP). In one example, the amphiphilic block copolymer comprises a poly(styrene) (PS) block and a PEG (or PEO) block. In such an example, the amphiphilic block copolymer comprises a PS block having a number-average molecular weight in the range between about 0.5 and 500 kDa and a PEG block having a number-average molecular weight in the range between about 0.5 and 500 kDa. The same molecular weight range is deemed applicable to other types of block copolymers mentioned above. In any of the block copolymers contemplated herein, e.g., whether it is PS-PEG or poly(t-butyl methacrylate) (PtMBA)-PEG, it is desirable to have a hydrophilic moiety (e.g., a hydroxyl (—OH) group) at the end of the PEG block rather than a hydrophobic moiety (e.g., a methoxy (—OCH₃) group). Examples of amphiphilic block copolymers contemplated herein include those shown in FIG. 2, wherein m (the degree of polymerization of the hydrophilic PEG block) is an integer in the range between about 11 and 11,000 and n (the degree of polymerization of the hydrophobic block) is an integer in the range between about 5 and 5,000. For example, each n can, independently, be an integer from about 5 to about 5,000. Alternatively, or in addition, each m is, independently, an integer from about 11 to about 11,000. For example, m can be from about 11 to about 2,200. In another example, n can be from about 5 to about 1,000. For example, m can be from about 11 to about 2,200. In another example, n can be from about 5 to about 1,000. For example, m can be from about 11 to about 5,500. In another example, n can be from about 5 to about 2,500.

The amphiphilic block copolymers contemplated herein can have a number-averaged molecular weight of the hydrophobic core block (M_{n,core}) of from about 2,000 g/mol to about 8,000 g/mol (e.g., from about 3,000 g/mol to about 6,000 g/mol, about 4,000 g/mol to about 8,000 g/mol, about 4,000 g/mol to about 7,000 g/mol, about 4,000 g/mol to about 6,000 g/mol, about 4,500 g/mol to about 5,500 g/mol or about 5,000 g/mol to about 5,500 g/mol); a number-averaged molecular weight of the PEG block (M_{n,PEG}) of from about 2,000 g/mol to about 8,000 g/mol (e.g., from about 3,000 g/mol to about 6,000 g/mol, about 4,000 g/mol to about 8,000 g/mol, about 4,000 g/mol to about 7,000 g/mol, about 4,000 g/mol to about 6,000 g/mol, about 4,500 g/mol to about 5,500 g/mol or about 5,000 g/mol to about 5,500 g/mol); and/or an overall molecular weight polydispersity (Mw/Mn) of from about 1.0 to about 2.0. For example, the overall molecular weight polydispersity can be from about 1.1 to about 2.0 or about 1.1 to about 1.5.

Each block of the amphiphilic block copolymers described herein can

The method of preparing an aqueous BCP micelle PLS solution using the equilibration nanoprecipitation (ENP) procedure comprises:
(a) dissolving a BCP (e.g., PS-PEG) in a water/organic cosolvent mixture at an initial organic cosolvent (e.g., acetone) composition in the range between about 10 and 90% (v/v);
(b) sonicating or mechanically agitating or heating the solution (e.g., for at least 2 min); and
(c) keeping the solution quiescently or under mild stirring (e.g., for at least half an hour) for equilibration.

In any of the methods described herein, the single-step dialysis procedure comprises:
(a) dialyzing the pre-equilibrated BCP micelle solution against an aqueous reservoir (e.g., for at least an hour); and
(b) replacing the aqueous reservoir with a fresh aqueous medium at least once during the dialysis period.

The methods described herein also include a lyophilization process of amphiphilic block copolymer micelles prepared by ENP. In one example, the lyophilization process comprises:
(a) freezing an aqueous BCP micelle PLS solution (e.g., at a lyophilizer chamber temperature of −60 °C) (“freezing”);
(b) sublimating crystalized water from the PLS solution (e.g., at a chamber temperature of −30 °C and a chamber pressure of 70 mTorr) until no further sublimation of water occurs (“primary drying”); and
(c) desorbing and vaporizing the supercooled water adsorbed to the polymer lung surfactant nanoparticles (i.e., BCP micelles) (e.g., at a chamber temperature of 25 °C and a chamber pressure of 70 mTorr) (“secondary drying”).

The aqueous BCP micelle PLS solution can be lyophilized without using any excipients (i.e., lyoprotectants or cryprotectants). The lyophilization process comprises three steps: freezing, primary drying, and secondary drying. The initial freezing is typically done at a suitably fast rate (e.g., −2 °C/min). Between the freezing and primary drying steps, the sample is heated at a reasonably fast rate, e.g., at 1 °C/min. Between the primary and secondary drying steps, the sample is heated at a reasonably fast rate, e.g., at 1 °C/min. The resulting lyophilized PLS composition can be used for treatment of acute respiratory distress syndrome (ARDS), acute lung injury (ALI), and neonatal respiratory distress syndrome (NRDS) in dry powder form or in liquid (solution) form after reconstitution in an aqueous medium. The reconstitution process typically comprises:
(a) dissolving the lyophilized BCP micelle PLS powder in an aqueous medium;
(b) shaking or vortexing or mechanically agitating or sonicating the dissolved PLS micelles until the solution becomes transparent, i.e., to ensure that the powder is broken up into its constituent nanoparticles (BCP micelles).

The disclosure also relates to compositions comprising dried amphiphilic block copolymer micelles having a mean hydrodynamic diameter in the range between about 5 nm to 1,000 nm (e.g., about 5 nm to about 500 nm, about 5 nm to about 100 nm, about 5 nm to about 50 nm, about 100 nm to about 250 nm, about 20 nm to about 175 nm, about 20 nm to about 50 nm, about 15 nm to about 30 nm, or about 25 nm to about 200 nm) in the original hydrated state, wherein the drying is done substantially without using any excipients (such as sugar, derivatives of sugar, or PEG homopolymers, including, e.g., sucrose, (2-hydroxypropyl)-β-cyclodextrin, and small-molecular weight PEG). Common excipients like saccharides (e.g., sucrose and (2-hydroxypropyl)-β-cyclodextrin) and small-molecular weight poly(ethylene glycol) (PEG) deactivate the surface activity of PLS.

As described herein, the amphiphilic block copolymer can comprise a hydrophobic block comprising a styrene monomer unit and a hydrophilic block comprising an ethylene glycol or ethylene oxide monomer unit. The amphiphilic block copolymer can comprise a PS block having a number-average molecular weight in the range of 0.5-500 kDa and a PEG block having a number-average molecular weight in the range of 0.5-500 kDa. In one example, the end group of the PEG block of the PS-PEG block copolymer is a hydroxyl group. The PS-PEG micelles have high dimensionless PEG corona chain grafting densities (σ_{PEG}). E.g., the values of the dimensionless PEG grafting density (σ_{PEG}) are in the range between about 5 and about 50 (e.g., about 5 to about 20, about 5 to about 15, about 10 to about 20, about 5 to about 8, about 7 to about 15 or about 8 to about 16).

The disclosure further provides a kit comprising compositions comprising dried solid amphiphilic block copolymer micelles described herein in a suitable container. The kit can further comprise an aqueous liquid in a suitable separate container. The kit can contain instructions on how to use the kit to, e.g., treat, among other conditions, pulmonary disorders. The pulmonary disorders contemplated herein include pulmonary disorders caused by at least either of the factors, deficiency or deactivation of endogenous lung surfactant in a subject (e.g., a mammal, such as a human). Examples of such disorders include acute respiratory distress syndrome (ARDS), acute lung injury (ALI), and neonatal respiratory distress syndrome (NRDS).

All patent publications and scientific publications cited herein are incorporated by reference herein in its entirety.

Values expressed in ranges should be interpreted in a flexible manner as include not only the numerical values explicitly recited as the limits of the range but also all individual numerical values and subranges encompassed within that range as if each numerical value and subrange are explicitly recited. For example, a range of “about 0.1% to about 5%” or “about 0.1 to 5%” should be interpreted to include not just about 0.1% and about 5%, but also all individual values (e.g., 1%, 2%, 3%, 4%, etc.) and sub-ranges (e.g., 0.1% to 0.5%, 1.1% to 2.2%, 3.3% to 4.4%, etc.) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an” or “the” are used to include one or more than one unless the context clearly dictates otherwise. The term “or” is used to refer to a nonexclusive “or” unless otherwise indicated. In addition, it is to be understood that the phraseology or terminology, employed herein and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all scientific publications, granted patents and patent application documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or less than about 0.0005%, or about 0%, or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not the limitation thereof and can include modification thereto and permutations thereof.

### EXAMPLES

The disclosure can be better understood by reference to the following examples which are offered by way of illustration. The disclosure is not limited to the examples given herein.

### Experimental Procedures and Materials

### Synthesis of Block Copolymers

PS(5.2 kDa)-PEG(5.5 kDA), referred as PS-PEG-OH, was custom synthesized by a commercial vendor (Polymer Source) via the sequential anionic polymerization of styrene and ethylene oxide monomers.

PS(5.2 kDa)-PEG(5.0 kDA), referred as PS-PEG-OCH₃, was synthesized via reversible addition-fragmentation (chain) transfer polymerization (RAFT) using a premade PEG monomethyl ether (PEG-OH, Mn=5.0 kDa, Sigma-Aldrich) as the starting material. 4-cyano-4-[(dodecylsulfanylthiocarbonyl) sulfanyl] pentanoic acid (CDTPA, Sigma-Aldrich) was used as the RAFT agent. CDTPA was conjugated to PEG-OH via Steglich esterification in the presence of 4-dimethylaminopyridine (Sigma-Aldrich) and N,N′-dicyclohexylcarbodiimide (Sigma-Aldrich) in dichloromethane (Sigma-Aldrich). The PEG-CDTPA product was purified by precipitation in a 1:1 (v/v) mixture of hexane (Sigma-Aldrich) and diethyl ether (Sigma-Aldrich) three times. After drying under vacuum, the purified PEG-CDTPA was co-dissolved with 2,2′-azobis(isobutyronitrile) (free radical initiator, Sigma-Aldrich) in 1,4-dioxane (Sigma-Aldrich). The solution was degassed with three freeze-pump-thaw cycles. Then, anhydrous styrene (Sigma-Aldrich), which was purified with activated alumina (Sigma-Aldrich), was added to the mixture to initiate the RAFT process. The polymerization reaction was run at 75 °C for 20 h and terminated by exposing the reaction mixture to air. The resulting PS-PEG-OCH₃ product was triply precipitated in a 1:1 (v/v) mixture of hexane and diethyl ether and dried under vacuum.

PtBMA (4.9 kDa)-PEG(5.0 kDA), referred as PtBMA-PEG-OCH₃, was also synthesized via RAFT. The synthesis procedure was identical to the procedure described above except that tert-butyl methacrylate (Sigma-Aldrich) was used as the monomer.

The molecular weight and molecular weight distribution properties of the block copolymers were characterized by ¹H NMR and GPC. The results are summarized in Table 1. The exact chemical structures of the three block copolymers used in this study are presented in FIG. 1.

TABLE 1

| | PEG block | M_{n, core} | M_{n, PEG} | |
|---|---|---|---|---|
| Sample name | end group | (g/mol) | (g/mol) | M_{w}/Mₙ^{a)} |
| PS-PEG-OH | —OH | 5,200 | 5,500 | 1.11 |
| PS-PEG-OCH₃ | —OCH₃ | 5,150 | 5,000 | 1.23 |
| PtBMA-PEG-OCH₃ | —OCH₃ | 6,080 | 5,000 | 1.19 |

^{a)}Overall molecular weight polydispersity (M_{w}/Mₙ) obtained from gel permeation chromatography (GPC).

The block co-polymers have the following general structures:

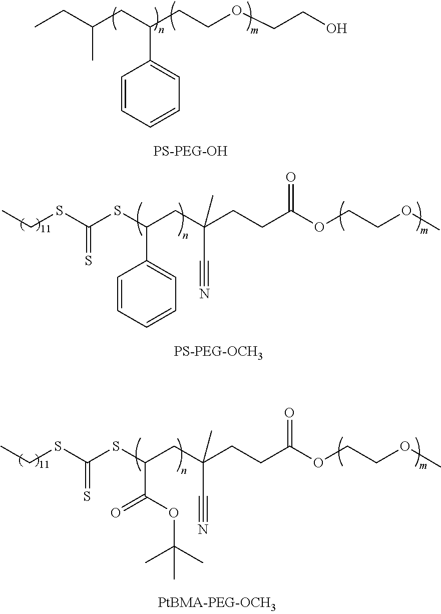

wherein m (the degree of polymerization of the hydrophilic PEG block) is an integer in the range between about 11 and 11,000 and n (the degree of polymerization of the hydrophobic block) is an integer in the range between about 5 and 5,000. For example, each n can, independently, be an integer from about 5 to about 5,000. Alternatively, or in addition, each m is, independently, an integer from about 11 to about 11,000. For example, m can be from about 11 to about 2,200. In another example, n can be from about 5 to about 1,000. For example, m can be from about 11 to about 2,200. In another example, n can be from about 5 to about 1,000. For example, m can be from about 11 to about 5,500. In another example, n can be from about 5 to about 2,500.

### PLS Formulation and Characterization

Block copolymer micelles with differing chemistry and sizes were prepared via a two-step solvent exchange technique termed as equilibrium nanoprecipitation (ENP) method. Each polymer (50 mg) was dissolved in a 5-mL mixture of acetone and Milli-Q water at varying solvent compositions (namely, the volume fraction of water, (φ_{w,ENP}), and each solution was sonicated for complete homogenization and subsequently equilibrated at ambient temperature for 24 hours. During the equilibration, block copolymers self-assemble into spherical micelles close to equilibrium whose size generally grows with φ_{w,ENP} probably owing to a greater selectivity of the solvent quality. To completely remove the acetone from the solution after the equilibration, the solution was transferred to a dialysis bag (Spectra/Por 7, 50 kDa molecular weight cutoff) in 1 L of water reservoir, and the dialysate was changed three times with fresh water over 48 hours. The final concentration of the aqueous micelle dispersion was adjusted to 5 mg/ml by adding fresh Milli-Q water.

Micelles formulated by the ENP method were characterized by transmission electron microscopy (TEM) using a 200-kV Tecnai T20 instrument. Diluted suspensions in Milli-Q water (0.1 mg/mL) were negatively stained by mixing in 1:1 ratio (v/v) with 1% aqueous uranyl acetate and then dried on a carbon-coated grid which was pre-treated by a glow discharge. The average diameter of micelles measured from the TEM images were regarded as the apparent core diameter (D_{c}), from which a non-dimensional grafting density of PEG (σ_{PEG}=pR_{g,PEG}²/D_{c}²), defined as the ratio of the cross-section area of a PEG chain (πR_{g,PEG}²) to the interfacial area per chain in the micelle (πD_{c}²/p), was calculated. R_{g,PEG} is the radius of gyration of an unperturbed PEG chain in water and is a function of weight-averaged PEG molecular weight. The term p=(πD_{c}³/6) (M_{n,core}/N_{A}ρ_{core})⁻¹ is the aggregation number (where M_{n,core} is the number-averaged molecular weight of the hydrophobic core block), N_{A} the Avogadro number, and ρ_{core} the density of the core block.

The hydrodynamic size (Dₕ) distribution, the z-average hydrodynamic size (<Dₕ>), and the polydispersity index (PDI) of micelle formulations before (“pristine”) and after the lyophilization-and-reconstitution process (“reconstituted”) were measured by dynamic light scattering (DLS) using a Brookhaven NanoBrook 90Plus instrument. The nanoparticle solution was diluted to 0.5 mg/mL in Milli-Q water and filtered using a poly(tetrafluoroethylene) (PTFE) syringe filter (pores of 0.45 μm). The DLS measurement was performed at a scattering angle of 90° and a laser wavelength of 640 nm. The micelle characteristics are summarized in Table 2.

TABLE 2

| | | | | | | Reconstituted after | | | |
|---|---|---|---|---|---|---|---|---|---|
| | Pristine | | | | | Lyophilization | | | |
| Polymer | φ_{w, ENP}^{a)} | D_{c} (nm) | σ_{PEG} ^{b)} | Dₕ (nm) | PDI | Dₕ (nm) | PDI | SIF ^{c)} | SA ^{d)} |
| PS-PEG-OH | 0.2 | 13.3 | 7.1 | 47.1 | 0.300 | 131.6 | 0.158 | 2.79 | 0.36 |
| | 0.3 | 17.0 | 9.0 | 29.4 | 0.220 | 131.7 | 0.148 | 4.48 | 0.23 |
| | 0.5 | 19.3 | 10.2 | 31.2 | 0.082 | 59.2 | 0.151 | 1.90 | 0.87 |
| | 0.6 | 21.3 | 11.3 | 35.6 | 0.112 | 69.7 | 0.166 | 1.96 | 0.81 |
| PS-PEG-OCH₃ | 0.1 | 14.8 | 8.0 | 26.8 | 0.177 | 152.1 | 0.141 | 5.68 | 0.09 |
| | 0.2 | 18.4 | 9.9 | 26.3 | 0.098 | 160.6 | 0.091 | 6.11 | 0.30 |
| | 0.3 | 18.9 | 10.2 | 28.8 | 0.029 | 158.9 | 0.170 | 5.52 | 0.25 |
| | 0.5 | 19.5 | 10.5 | 168.1 | 0.190 | 193.5 | 0.121 | 1.15 | ~4 |
| PtBMA-PEG- | 0.1 | 16.5 | 7.8 | 32.0 | 0.218 | 155.5 | 0.189 | 4.86 | 0.14 |
| OCH₃ | 0.2 | 17.4 | 8.3 | 26.1 | 0.114 | 152.9 | 0.188 | 5.86 | 0.13 |
| | 0.3 | 19.9 | 9.4 | 27.5 | 0.082 | 109.6 | 0.133 | 3.99 | 0.18 |
| | 0.5 | 23.3 | 11.1 | 91.2 | 0.284 | 168.2 | 0.212 | 1.84 | 0.45 |

^{a)}Volume fraction of water used in the equilibrium nanoprecipitation (ENP) method.
^{b)} Nondimensionalized PEG grafting density.
^{c)} Size increasing factor, i.e., hydrodynamic diameter ratio of reconstituted micelles to pristine micelles.
^{d)} Surface availability.

### Lyophilization and Reconstitution

Each micelle formulation (2 mL) was transferred into a 10-mL lyophilization vial equipped with a rubber stopper. A total of 12 vials (3 polymers×4 ENP conditions) were placed together in the chamber of a small-batch lyophilizer (MicroFD, Millrock Tech) with programmable thermal trajectories. The lyophilization process followed a typical sequence; the solutions were rapidly cooled from 25 °C to the target shelf temperature (T_{shelf}) and then allowed to equilibrate for 3 h (the freezing stage). Subsequently, the frozen water (ice) was sublimated at a T_{shelf} below the “collapse” temperature of the PEG/water system (−18 °C) until the chamber vapor pressure (P) reached the target pressure of 70 mTorr (the primary drying stage). Finally, the bound water was dried at a T_{shelf} above the triple point of water (0.01 °C) until the target pressure of P=70 mTorr was achieved again (the secondary drying stage). Two different operating methods with distinct ramping rates and target T_{shelf} values were utilized, and the specific operational parameters can be found in Table 3. Throughout the lyophilization process, T_{shelf}, P, and the heat flux between the vials and the shelf were continuously monitored in real-time. In reconstitution, 2 mL of Milli-Q water was added to each vial containing lyophilized samples, followed by gentle shaking for 3 min and sonication for 3 min.

TABLE 3

| | | | | Primary Drying | | Secondary Drying | | Reconstituted after | | |
|---|---|---|---|---|---|---|---|---|---|---|
| | | Freezing Stage | | Stage | | Stage | | Lyophilization | | |
| | Formulation | R | T_{shelf} | R | T_{shelf} | R | T_{shelf} | <Dₕ> | | |
| Method | Volume (mL) | (°C/min) ^{a)} | (°C) ^{b)} | (°C/min) ^{a)} | (°C) ^{b)} | (°C/min) ^{a)} | (°C) ^{b)} | (nm) | PDI | SIF ^{c)} |
| A | 38 | −2 | −60 | +2 | −30 | +1 | 25 | 69.5 | 0.18 | 1.95 |
| B | 12 | −1 | −50 | +1 | −35 | +1 | 25 | 82.3 | 0.17 | 2.40 |

^{a)} Cooling or heating rate.
^{b)} Shelf temperature.
^{c)} Size increasing factor.

### Dynamic Light Scattering (DLS)

An aqueous BCP micelle solution (0.5 mL of a 5 mg/mL) was diluted by adding 2.5 mL of Milli-Q-purified water. The diluted solution was filtered using a poly(tetrafluoroethylene) (PTFE) filter with 0.45 μm pore size twice to remove dusts, bubbles, etc. The filtrate was transferred to a polystyrene disposable cuvette for DLS measurement. The mean hydrodynamic diameter, polydispersity, and zeta potential of the BCP micelles were determined by DLS using a NanoBrook ZetaPALS instrument (Brookhaven, illumination with a 659 nm laser, detection at 90° scattering angle).

### Transmission Electron Microscopy (TEM)

A TEM specimen was prepared as follows. For negative staining, 5 μL of a 1 mg/mL aqueous BCP micelle solution was mixed with 5 μL of a 1% aqueous uranyl acetate solution. The mixed solution was placed on a carbon-coated copper TEM grid (treated with O₂ plasma to make the surface hydrophilic). After 30 seconds, the solution was blotted using filter paper and dried. The resulting specimen was imaged using a 200-kV Tecnai T20 TEM instrument. The TEM images were analyzed using Gatan DigitalMicrograph software.

### Surface Pressure—Area Isotherms (Surface Mechanical Analysis)

The surface pressure-area (Π-A) isotherms of the micelle monolayers were measured using a Langmuir trough (KSV 5000, Biolin scientific) with two symmetric barriers attached at each end. The trough and two barriers were initially cleaned with ethanol and water three times. Water (1.4 L of Milli-Q purified water) was poured in the trough, and filter paper was used as a Wilhelmy plate to measure the surface tension. Before each measurement, the water surface was aspirated to remove any interfacial contaminants so that the surface pressure of pure water at the maximum compression (A=71 cm²) did not exceed 0.2 mN/m.

Onto the clean surface of water at the maximum area (782 cm²), 100 μL of the “pristine” or “reconstituted” micelle solution (5 mg/mL) was spread using a microsyringe. Droplets of a few microliter was formed on a tip of the syringe needle and carefully contacted on the water surface to spread the sample on the water. For the Π-A isotherm measurements of the PLS with added excipients, excipients were added to the pristine solutions to reach a 10% w/v excipient concentration before the spreading. For the Π-A isotherms of the solid lyophilizates, 1.5 mg of the foamy solid was carefully placed piecewise on the clean water surface using a dressing forcep.

### Differential Scanning Calorimetry

Freezing and melting of the micelle formulations were analyzed using differential scanning calorimetry (DSC; DSC 4000, Perkin Elmer). The micelle concentration of ~100 mg/ml (i.e., ~20 times higher than the concentration of standard formulations) was used to precisely evaluate the amount of bound water to the PEG brushes. To increase the concentration, each of pristine micelle formulation (0.5 mL, 5 mg/mL) was placed in a pre-rinsed centrifugal dialysis membrane tube (Amicon Ultra-0.5, MWCO 10 kDa) and centrifuged at 14,000 rpm for 20 min. The micelle concentration post centrifugation was measured using GPC by taking an aliquot of the concentrated micelle suspension. The GPC intensity peak area was converted to the concentration based on a calibrated data (a standard solution of predetermined polymer concentration). The actual concentrations of micelle suspensions used in DSC are reported in Table 4.

A concentrated solution (8 μL) was transferred to a hermetic DSC pan and tightly sealed. A DSC cycle of cooling from 30 °C to −50 °C and reverse heating back to 30 °C was performed with a ramp rate of 5 °C/min. The mass of eutectic PEG (m_{PEG,eu}) was calculated from the measured endothermic peak area (Qₑᵤ) at the eutectic point according to:

Qₑᵤ=mₑᵤΔHₑᵤ=(m_{PEG,eu}/w_{PEG,eu})ΔHₑᵤ (1)

where ΔHₑᵤ=142±1 J/(g eutectic mixture) was evaluated by linear fits of the enthalpies of fusion of the PEG/water eutectic mixtures and w_{PEG,eu}=0.47 is the mass fraction of PEG at the eutectic point (Tₑᵤ=−19 °C). Given that a portion of PEG segments are dehydrated to cover the surface of hydrophobic core, only the rest (e.g., hydrated PEG segments) can form a eutectic mixture with nearby water. The fraction of hydrated PEG, f_{hyd}=m_{PEG,eu}/m_{PEG}, was evaluated from eq 2:

ΔH_{eu:PEG}=Qₑᵤ/m_{PEG}=[m_{PEG,eu}/(w_{PEG,eu}m_{PEG})]ΔHₑᵤ=(f_{hyd}/w_{PEG,eu})ΔHₑᵤ, (2)

where m_{PEG} is the total mass of PEG in the sample, and ΔH_{eu:PEG} is the enthalpy of fusion at the eutectic point per total mass of PEG. ΔH_{eu:PEG} and f_{hyd} for each sample are reported in Table 4.

TABLE 4

| | | | T_{f} | Tₑᵤ | ΔH_{eu:PEG} | | Tₘ |
|---|---|---|---|---|---|---|---|
| Polymer | φ_{w, ENP}^{a)} | w_{p, analyte} ^{b)} | (°C) ^{c)} | (°C) ^{d)} | (J/g PEG) ^{d)} | f_{hyd} ^{e)} | (°C) ^{f)} |
| PS-PEG-OH | 0.2 | 0.10 | −15.6 | −15.6 | 38 | 0.12 | 8.7 |
| | 0.3 | 0.08 | −15.1 | −16.5 | 28 | 0.09 | 8.4 |
| | 0.5 | 0.09 | −13.7 | −16.3 | 50 | 0.16 | 8.1 |
| | 0.6 | 0.06 | −12.6 | −15.9 | 84 | 0.27 | 8.9 |
| PS-PEG-OCH₃ | 0.1 | 0.03 ^{g)} | −13.3 | | 0 | 0 | 9.1 |
| | 0.2 | 0.02 ^{g)} | −12.4 | −15.1 | 110 | 0.37 | 7.1 |
| | 0.3 | 0.04 ^{g)} | −19.6 | −15.5 | 66 | 0.22 | 9.0 |
| | 0.5 | 0.01 ^{g)} | −18.0 | −14.6 | 174 | 0.58 | 8.0 |
| PtBMA-PEG-OCH₃ | 0.1 | 0.08 | −17.1 | | 0 | 0 | 10.3 |
| | 0.2 | 0.09 | −13.7 | | 0 | 0 | 9.3 |
| | 0.3 | 0.07 | −10.5 | | 0 | 0 | 8.6 |
| | 0.5 | 0.08 | −19.8 | −15.6 | 45 | 0.17 | 8.8 |

^{a)}Volume fraction of water used in the equilibrium nanoprecipitation (ENP) method.
^{b)} Weight fraction of polymer in the concentrated suspension (prepared via centrifugation) determined via chromatography.
^{c)} Freezing point.
^{d)} Eutectic peak melting point and the enthalpy of fusion per gram of PEG in the system.
^{e)} Hydrated PEG fraction, defined as the ratio of the PEG segments contained in the eutectic mixture to the total PEG in the system.
^{f)} Melting point of the bulk water.
^{g)} Aggregation of micelles leading to loss of micelles in the concentrated suspension (prepared via centrifugation).

### Results and Discussion

### Surface Mechanics of PS-PEG-OH Micelles in the Presence of Excipients

Excipients are commonly added to prevent structural damage and irreversible aggregation of nanoparticulate drugs caused by freezing and drying stresses, which result from ice crystallization and solvent evaporation, respectively, in conventional lyophilization processes. These excipients are typically hydrophilic molecules that can interact favorably with both water (through hydrogen bonding) and particle surface (through van der Waals interactions). However, due to this amphiphilic nature of excipients, they can have detrimental effects on the surface activity of block copolymer micelle formulations, either by modifying the surface energy of hydrophobic micellar cores or by competitively adsorbing at the air-water interface. To examine the effect of excipients on the surface mechanical behavior of the model PLS formulation prior to the lyophilization study, two saccharides (sucrose and HPβCD) and two hydrophilic polymers (2 and 5 kg/mol PEG-OCH₃) were selected as representative excipients. A concentration of 10% w/v, which falls within the typical range of 5-20% w/v for optimal protection, was used.

FIG. 1 compares the surface pressure-area (Π-A) isotherms of PS-PEG-OH micelles formulated at φ_{w,ENP}=0.6 with and without excipients. Here, the surface pressure (Π=γ₀−γ), defined as the decrement in surface tension from that of pure water (γ₀=72 mN/m), is plotted against the trough area (A). The chosen model formulation exhibited the ability to achieve low surface tension (γ<10 mN/m) upon compression of the surface, as demonstrated by the control isotherm. Strikingly, the addition of any excipient to the PS-PEG-OH formulation resulted in the loss of surface activity, preventing the attainment of high Π values. While not wishing to be bound by any specific theory, it is speculated that saccharides and PEG-OCH₃ excipients reduce the surface activity of PS-PEG-OH micelles in different ways. Consistent with a previous study, the isotherms of the saccharide-containing formulations resemble the typical surface mechanical behavior of block copolymer micelles with weakly hydrophobic cores, such as poly(D,L-lactic acid-block-ethylene glycol) (PLA-PEG). This suggests a possible reduction in the interfacial tension between water and the hydrophobic PS core due to the mediating effect of saccharides, in line with literature on the favorable interactions between sucrose and globular proteins.

On the other hand, PEG-OCH₃ excipients compete with the adsorption of PS-PEG-OH micelles at water surfaces. The maximum Π values of PEG(2 and 4.6 kg/mol) monolayers formed by spontaneous adsorption from the subphase were 8 and 9 mN/m, respectively, which are nearly the same as the equilibrium spreading pressure of PS-PEG-OH micelles (10 mN/m). Thus, PEG-OCH₃ excipients (at a weight ratio of 20:1 to PS-PEG-OH micelles) likely occupy most of the available surface area and reduce the amount of adsorbed PS-PEG-OH micelles at the water surface. Apparently, it is difficult to use excipients as cryo- and lyoprotectants without causing the reduction in surface activity of PLS. Moreover, the removal of excipients from lyophilized samples requires complicated purification steps, such as prolonged dialysis, which is not clinically practical.

### Operation Parameters for Excipient-Free Lyophilization of PS-PEG-OH Micelles

To avoid the problems in using excipients, an aim was to investigate the optimal operating conditions for excipient-free lyophilization. The same formulation as in the section entitled Surface Mechanics of PS-PEG-OH Micelles in the Presence of Excipients (PS-PEG-OH micelles formulated at φ_{w,ENP}=0.6) was used because it exhibited the highest PEG grafting density (σ_{PEG}) among all formulations in this study (Table 3; the effect of σ_{PEG} will be discussed later).

To identify the impact of process parameters on the quality of excipient-free lyophilization, two different methods were employed using a small-batch research-scale lyophilizer (MicroFD, Millrock Tech). Method A was designed to achieve more aggressive freezing compared to Method B, characterized by a faster cooling rate and a lower shelf temperature (T_{shelf}) during the freezing stage (refer to Table 3 for the operation parameters of Methods A and B). FIGS. 2A and 2B depict the heat flux evolution between the shelf and the vial chamber along the initial freezing trajectory, where a negative peak in the heat flux corresponds to ice crystallization. The onset of the heat flux peak, indicating ice nucleation, occurred at a lower shelf temperature (−27.2 °C) for Method A compared to Method B (−21.4 °C). The lower nucleation temperature leads to higher nucleation density and smaller crystallites, which can reduce the mechanical stress exerted by ice crystals and thus benefit nanoparticle preservation. Additionally, the interval between ice nucleation and the maximum rate of ice growth (indicated by shaded regions in FIGS. 2A-2B was slightly shorter (9 min) for Method A compared to Method B (10 min), suggesting slightly faster crystallization when the cooling rate is doubled.

On the other hand, during the primary drying stage, both Methods A and B underwent steady-state sublimation of water until complete evaporation. Since the T_{shelf} values used in the primary drying stage were significantly lower than the eutectic temperature (Tₑᵤ=−19 °C) and the “collapse” temperature (−18 °C) in both methods, the quality of lyophilization (as discussed below) is unlikely to be influenced by the differences in primary drying stage parameters.

Consequently, Method A yielded better redispersibility and surface activity of the model formulations after reconstitution compared to Method B. The size increasing factor (SIF), defined as the ratio of the z-average hydrodynamic diameter (<Dₕ>) of micelles after reconstitution to that before lyophilization, was lower for Method A (Table 3), indicating less pronounced irreversible aggregation. Additionally, the difference between the I-A isotherms of pristine and reconstituted micelles from Method A (FIG. 2C) was smaller than that from Method B (FIG. 2D), although both reconstituted micelles could achieve high surface pressure (Πₘₐₓ≅70 mN/m) at maximum surface compression. Therefore, Method A was selected as the standard lyophilization procedure for the subsequent experiments.

In polymer-grafted nanoparticles, the grafting density of hydrophilic polymers (σ_{PEG}) is a critical parameter to the colloidal stability of the formulations. A higher σ_{PEG} leads to a thicker and denser PEG brush, promoting stable dispersion. This stabilizing effect of densely grafted polymer brushes is particularly relevant during the freezing stage, where ice growth reduces the available space for the particles and forces them to come into close proximity (referred to as “cryo-concentration”). In such situations, a high σ_{PEG} is expected to protect the particles from irreversible aggregation by efficiently dampening the compressive stress. This is because the PEG brush remains partially hydrated and amorphous even below freezing temperature (see below for detailed discussion).

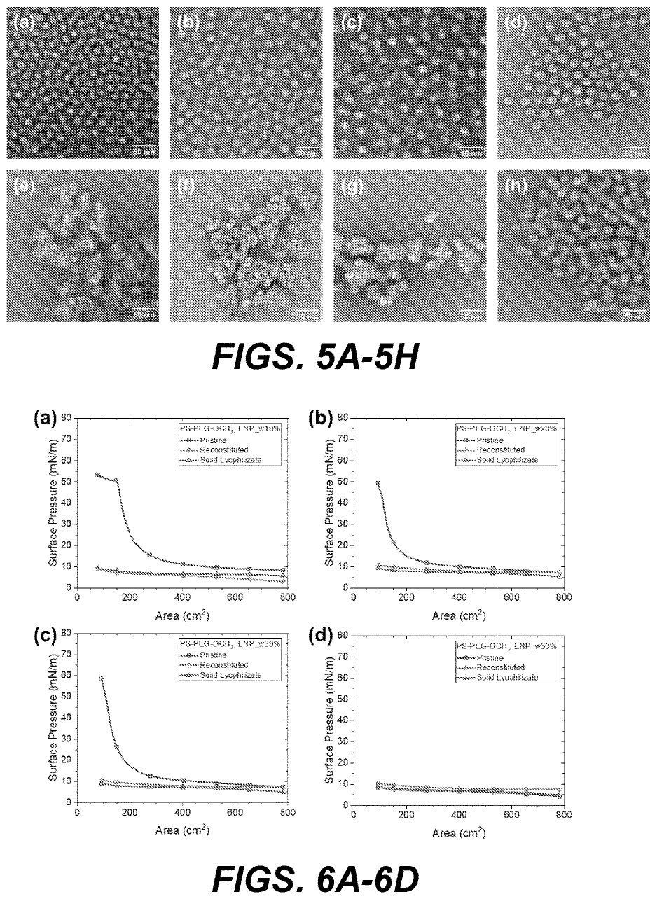

It is noteworthy that Logie et al. have observed enhanced redispersibility following lyophilization, utilizing nonlinear (graft) copolymers that incorporate a higher number of PEG side chains, without the need for excipients. But, in the examples provided by Logie et al., the mean hydrodynamic diameters of the polymer micelles consistently exceeded approximately 85 nm. In contrast, as indicated in Table 2 above, the majority of the micelles described therein exhibited hydrodynamic diameters of less than approximately 50 nm. And there is evidence that the therapeutic effectiveness of micelles, particularly as polymer lung surfactants, can be compromised when the mean hydrodynamic diameter exceeded approximately 55 nm. See, e.g., Kim et al., ACS. Appl. Bio. Mater., 2018, (FIG. 5), which is incorporated by reference as if fully set forth herein. Accordingly, the disclosure relates to micelles with a mean hydrodynamic diameter of less than about 90 nm, less than about 85 nm, less than about 80 nm, less than about 75 nm, less than about 70 nm, or less than about 60 nm; such as from about 1 nm to about 90 nm, about 10 nm to about 80 nm, about 10 nm to about 75 nm, about 20 nm to about 80 nm, about 15 nm to about 60 nm, or about 25 nm to about 90 nm.

In the systems described herein, the σ_{PEG} was systematically controlled by varying the number of block copolymer chains that constitute a micelle, also known as the aggregation number (N_{agg}). This control was achieved by applying the equilibrium nanoprecipitation (ENP) method with different qualities of a selective solvent. A higher water fraction in the ENP medium (φ_{w,ENP}) results in self-assembly with a higher N_{agg} during the process of free energy minimization, leading to larger σ_{PEG}. In this study, four micelle formulations were tested with different σ_{PEG} values ranging from 7.1 to 11.3. These formulations were prepared using the identical starting polymer but at different φ_{w,ENP} values (Table 2). The pristine micelles exhibited uniform sizes in all formulations, and the average core diameter (D_{c}) increased monotonically with φ_{w,ENP}. However, the z-average hydrodynamic radius (<Dₕ>) of the pristine micelles did not follow a monotonic trend with φ_{w,ENP} due to an outlier (φ_{w,ENP}=0.2) with the presence of significant amounts of large clusters (Table 2).

The redispersibility of micelles after lyophilization and subsequent reconstitution showed a significant increase with σ_{PEG}. Transmission electron microscopy (TEM) images of the reconstituted micelles revealed that clustered micelles were larger than 100 nm at φ_{w,ENP}=0.2 and 0.3, but decreased to approximately 50 nm at higher φ_{w,ENP} values (FIGS. 3E-3H). Dynamic light scattering (DLS) analysis provided consistent results, as indicated by the unanimous increase in the z-average hydrodynamic radius (<Dₕ>) after lyophilization (Table 2). However, the size increasing factor (SIF), defined as the ratio of <Dₕ> of reconstituted micelles to pristine micelles, decreased with increasing φ_{w,ENP} or, equivalently, increasing ØPEG.

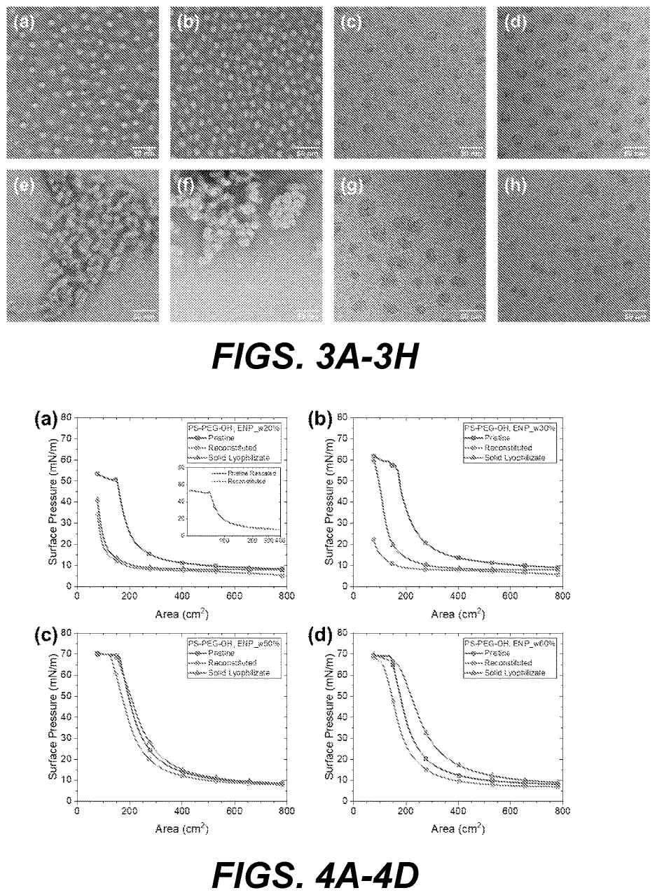

The surface activity of the reconstituted micelles exhibited a strong correlation with their redispersibility, indicating that it was preserved more effectively with higher σ_{PEG}. FIGS. 4A and 4B demonstrate that the surface pressure (Π) was significantly reduced at φ_{w,ENP}=0.2 and 0.3. This reduction can be attributed to the low availability of surface-active micelles, as clustered micelles with irregular shapes cannot expose their hydrophobic cores to the air efficiently and therefore are easily detached from the air/water surface. To quantify this effect, the concept of surface availability (SA) was introduced, which represents the multiplication factor required to overlap the pristine isotherm with the reconstituted isotherm (inset in FIG. 4A). For example, an SA value of 0.36 at φ_{w,ENP}=0.2 indicates that only 36% of surface-active micelles from the reconstituted formulation are available. As the micelles become more densely grafted with PEG, the Π-A isotherms of the reconstituted formulations approach those of the pristine micelles (FIGS. 4D-4E), resulting in an increase in SA close to unity (Table 2). This clearly demonstrates the feasibility of excipient-free lyophilization when the pharmaceutical formulation consists of densely PEG-grafted micelles.

Furthermore, the lyophilized micelles in powder form could also be spread on the water surface and exhibited surface activity even without reconstitution. The Π-A isotherms of solid lyophilizates were compared with those of the reconstituted formulations in FIGS. 4A-4E. It was observed that the solid lyophilizates displayed comparable or even stronger surface activity, partially due to a larger amount of material deposited on the surface (approximately 1.5 mg of solid was deposited, theoretically three times greater than the mass of solute used in the pristine and reconstituted aqueous formulations). Nevertheless, the feasibility of directly spreading solid lyophilizates to achieve high surface pressure (Π) is encouraging, as it may enable the delivery of PLS micelles to the alveolar space (deep inside the lungs) through inhalation, which is a more desirable approach than liquid instillation in clinical practice.

### Effects of End Group and Core Block Chemistry

The study was expanded to include an analogous amphiphilic block copolymer, PS-PEG-OCH₃, which has similar block molecular weights and high surface activity. The main difference between PS-PEG-OH and PS-PEG-OCH₃ is the end group chemistry at the PEG block (Table 1). Similar to the PS-PEG-OH series, we prepared micelles from PS-PEG-OH and PS-PEG-OCH₃ using the ENP method with four (4) different φ_{w,ENP}. TEM images confirmed that all micelles were uniformly sized (FIGS. 5A-5H) and exhibited higher values of σ_{PEG} with increasing (φ_{w,ENP}. However, DLS analysis revealed an unusually large <Dₕ> for the φ_{w,ENP}=0.5 formulation, indicating the presence of supra-micellar aggregates (Table 2). Additionally, the micelles formulated at φ_{w,ENP}=0.5 showed minimal surface activity (FIG. 6D), making them unsuitable as PLS.

Reconstituted PS-PEG-OCH₃ micelles after lyophilization appeared as large chunks (>100 nm) in all samples (FIGS. 5E-5H). DLS analysis showed higher size increasing factors (SIF) for PS-PEG-OCH₃ micelles (SIF>5), except for the φ_{w,ENP}=0.5 formulation, which already had large, aggregated clusters in the pristine state. In comparison, the SIF values for PS-PEG-OH micelles were generally smaller (SIF<5) (Table 2). These systematic differences indicate that a small change in the end group chemistry of the PEG block, from hydroxy to methoxy, leads to a significantly decreased stability. While not wishing to be bound by any specific theory, this may be attributed to the bridging effect of the hydrophobic methoxy (—OCH₃) end group, which promotes closer contact between the micellar cores, especially under cryo-condensed conditions. It is interesting to note that significant aggregation of all PS-PEG-OCH₃ micelles also occurred after centrifugation (at approximately 11,000×g for 20 min), whereas the centrifuged PS-PEG-OH micelles were easily redispersed (Table 4). Furthermore, both reconstituted and solid lyophilized forms of PS-PEG-OCH₃ micelles exhibited impaired surface activity (FIGS. 6A-6D), and the surface availability (SA) after reconstitution was less than 0.3 in all samples, except for the φ_{w,ENP}=0.5 formulation (Table 2).

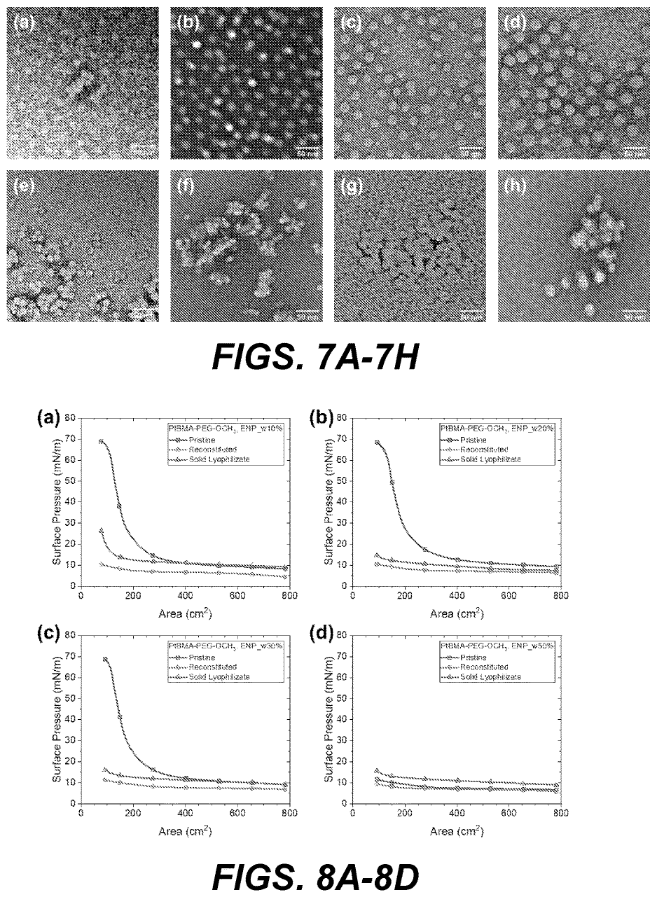

Next, another amphiphilic block copolymer, PtBMA-PEG-OCH₃, with a different core block, was investigated for excipient-free lyophilization. In its pristine form, the PtBMA-PEG-OCH₃ micelle formulation exhibited high surface activity, generating surface pressures as high as/7=68 mN/m in our recent study. TEM images confirmed that all PtBMA-PEG-OCH₃ micelles formulated at 4 different φ_{w,ENP} appeared as uniformly sized particles (FIGS. 7A-7D). However, for the φ_{w,ENP}=0.5 formulation, an unusually large <Dₕ> and the inability to generate high surface pressure upon compression indicated the presence of supra-micellar aggregates (Table 2 and FIGS. 8A-8D).

Due to the hydrophobic methoxy end group at the PEG chain of PtBMA-PEG-OCH₃, the lyophilization results were similar to those of PS-PEG-OCH₃ micelles. Reconstituted PtBMA-PEG-OCH₃ micelles after lyophilization were observed as large chunks (>100 nm) (FIGS. 7E-7H). DLS analysis consistently showed size increasing factors (SIF) greater than 3 for reconstituted micelles, except for the φ_{w,ENP}=0.5 formulation where the pristine micelles already exhibited large superstructures (Table 2). Once again, the pronounced aggregation in PtBMA-PEG-OCH₃ micelles after excipient-free lyophilization can be primarily attributed to the bridging effect of the methoxy (—OCH₃) end group. Consequently, the surface activity of PtBMA-PEG-OCH₃ micelles in both reconstituted and solid lyophilized forms was compromised (FIGS. 8A-8D). Furthermore, the surface availability (SA) after reconstitution was below 0.2 in all samples, except for the φ_{w,ENP}=0.5 formulation (Table 2). However, there were slight differences in the lyophilization results between PS-PEG-OCH₃ and PtBMA-PEG-OCH₃ micelles (excluding all φ_{w,ENP}=0.5 formulations). The overall SIF values were smaller, and the solid lyophilizates exhibited higher surface activity in PtBMA-PEG-OCH₃ micelles. While not wishing to be bound by any specific theory, this may suggest that PtBMA-PEG-OCH₃ micelles may be slightly more stable in maintaining colloidal dispersion. Similarly, the centrifuged PtBMA-PEG-OCH₃ micelles did not aggregate as strongly as the PS-PEG-OCH₃ micelles. However, the exact origin of this difference (in relation to the core block chemistry) is not yet fully understood.

### Differential Scanning Calorimetry (DSC)

The stability of PS-PEG-OH micelles increased as the grafting density (σ_{PEG}) grew, whereas no such effect was observed in PS-PEG-OCH₃ and PtBMA-PEG-OCH₃ micelles. To further investigate the impact of grafting density during the lyophilization cycle, we measured DSC thermograms of the aqueous micelle solutions. The solutions were cooled from 30 °C to −50 °C and subsequently heated to 30 °C at a ramping rate of +5 °C/min (FIGS. 9A-9C). To accurately capture the PEG/water eutectic behavior (Tₑᵤ=−19 °C), the solution concentrations were increased through centrifugal dialysis. The polymer weight fraction after centrifugation (ω_{p,analyte}) ranged from 1% to 10% (Table 4). Notably, significant aggregation occurred in PS-PEG-OCH₃ micelles formulated at φ_{w,ENP}=0.1, 0.2, and 0.5, resulting in a substantial loss of micelles in the concentrated suspensions.

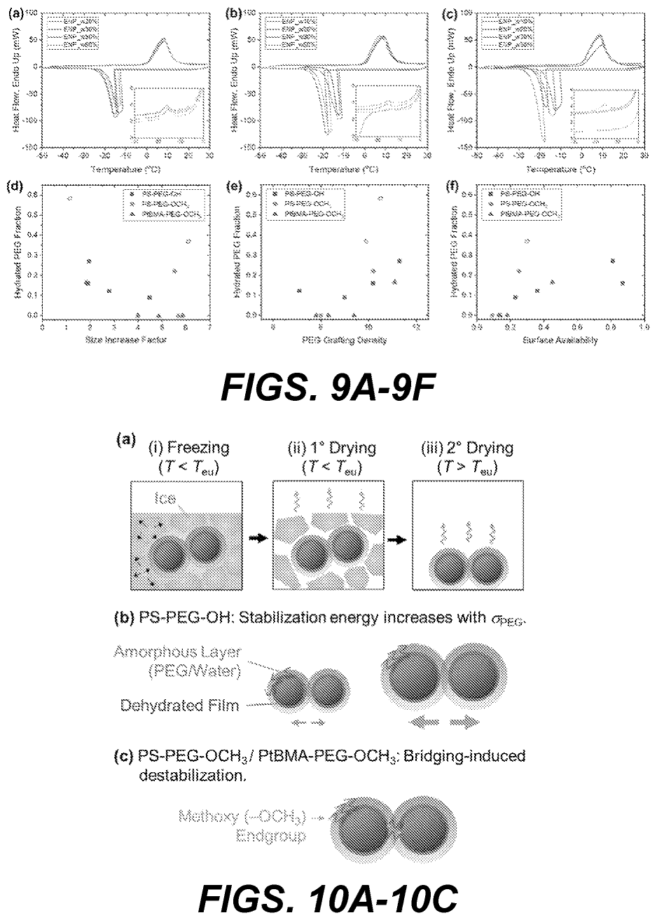

DSC thermograms provided insights into the nanoscopic changes of PEG-grafted micelles in the lyophilization medium (FIG. 10A). Typically, phase separation between PEG and water occurs after the bulk ice crystallization at the freezing point (Tₜ). However, as described in previous studies, not all PEG and water components separate into perfectly isolated crystals; instead, a substantial amount of amorphous PEG/water mixture remains even below Tₑᵤ. Upon heating the system from −50 °C to Tₑᵤ, the crystallized portion of PEG and the adjacent water melt to form a eutectic mixture. This eutectic melting is displayed in the enlarged thermograms (insets of FIGS. 9A-9C). The Tₑᵤ values were in close agreement (−15.5±0.9 °C), but the specific heat of fusion for the eutectic mixture (ΔH_{eu:PEG}) varied among the samples (Table 4). Moreover, PS-PEG-OCH₃ (φ_{w,ENP}=0.1) and PtBMA-PEG-OCH₃ (φ_{w,ENP}=0.1, 0.2, and 0.3) micelles did not exhibit eutectic melting. After surpassing Tₑᵤ, the bulk ice finally melted at the melting point (Tₘ). Most of the samples showed very similar Tₘ values and specific heat of melting.

The eutectic melting was analyzed to evaluate the fraction of PEG segments that can freely interact with water, referred to as the hydrated PEG fraction (f_{hyd}). The distinction between hydrated and dehydrated PEG segments arises from the presence of a hydrophobic surface in block copolymer micelles. Previous studies have demonstrated that PEG brushes can form dense and nearly dehydrated layers at solid-water interfaces to reduce surface energy. It may be that strongly hydrophobic PS and PtBMA cores necessitate partial dehydration of PEG brushes to form a dense layer (FIGS. 10A-10C). The remaining PEG segments can be hydrated, and when a micellar suspension is frozen below Tₑᵤ, a portion of the hydrated PEG crystallizes, while the other portion remains amorphous as a supercooled mixture with water. The measured heat of fusion at Tₑᵤ (ΔH_{eu:PEG}) originates from the cooperative melting of the crystalline portion of PEG in contact with ice, and it is suppressed when the PEG brush is less hydrated, less hydrated PEG segments crystallize during the freezing stage, or the crystalline PEG segments lose contact with water. Therefore, ΔH_{eu:PEG} provides an easy way to characterize the state of PEG brushes in the frozen micelle solution.

In the reference system (an aqueous solution of homopolymer PEG), the empirical heat of melting per PEG/water eutectic mixture (ΔHₑᵤ) was 142 J/(g eutectic mixture). Note that this value is significantly lower than the theoretical limit of ~270 J/(g eutectic mixture) indicating imperfect crystallization. Owing to the similar DSC methods (rapid cooling) used in the present work and the reference (−5 °C/min), a comparable PEG crystallinity with the reference system can be assumed. Thus, we used eq. 2 with ΔHₑᵤ=142 J/(g eutectic mixture) to evaluate f_{hyd} of the micelle samples. The results of f_{hyd} (Table 4) show that, in most samples, the majority of PEG segments exists as a dehydrated film to cover the hydrophobic core surface (i.e., f_{hyd}<0.5). Nevertheless, it is rather surprising that PS-PEG-OCH₃ (φ_{w,ENP}=0.3) and PtBMA-PEG-OCH₃ (φ_{w,ENP}=0.1, 0.2, and 0.3) micelles lacks any signature of eutectic melting. While not wishing to be bound by any specific theory, it may be that freezing-induced massive aggregation in these samples isolated most of the PEG segments within the interior of aggregated micellar superstructure. Such an entrapping effect may have prevented the crystallized portion of PEG from eutectic melting with water. The correlation between f_{hyd} and the size increase factor (SIF) obtained from the lyophilization experiment (FIGS. 9A-9D) supports this idea; generally, f_{hyd} is lower when the sample has a greater SIF (e.g., intense aggregation).

Regarding the effect of micelle structure, FIG. 9E illustrates a clear trend of increasing f_{hyd} with higher σ_{PEG}. In PS-PEG-OH micelles, the hydrated fraction gradually increases as σ_{PEG} increases. Since the area per PEG chain required to cover the core surface decreases with increasing σ_{PEG}, the PEG chains can allocate more segments to be hydrated after forming a dehydrated film (FIG. 10B). Consequently, micelles with higher σ_{PEG} exhibit thicker amorphous layers, which can effectively mitigate freezing-induced stress during the freezing stage. Similarly, in PS-PEG-OCH₃ and PtBMA-PEG-OCH₃ micelles, the eutectic melting behavior is observed only at higher σ_{PEG}, indicating that the fraction of hydrated PEG is too low at lower σ_{PEG}. However, due to the presence of hydrophobic methoxy end groups, the PEG chains now act as linkers between the cores, leading to irreversible aggregation (FIG. 10C). This suggests that block copolymers with a hydrophobic end group in the PEG block are susceptible to destabilization during lyophilization. Therefore, excipient-free lyophilization is feasible only if (i) the PEG grafting density is sufficiently high to form an amorphous layer which can dissipate the external freezing stress and (ii) the PEG end group does not induce bridging interactions.

PEG grafting density and the endgroup at the PEG block simultaneously affect both the surface availability (SA) of the reconstituted micelles and the hydrated PEG fraction (f_{hyd}). Indeed, FIG. 9F reveals a strong correlation between f_{hyd} and SA even if the thermal trajectories used in the two experiments (DSC and lyophilization) were not the same. Since SA is the most important property in terms of pharmaceutical surfactant formulation, this finding suggests that DSC can serve as an initial screening tool for evaluating the success of excipient-free lyophilization of PLS.

### CONCLUSIONS

Although the use of excipients is a common practice in pharmaceutical lyophilization, there are instances where the removal of excipients becomes necessary. Polymer lung surfactant (PLS) are a case where commonly used excipients, such as saccharides and PEG, hinder the surface activity of PLS. Described herein are certain optimal conditions for excipient-free lyophilization, including material and operating parameters, using a small-batch research-scale lyophilizer. Micelles formulated through equilibrium nanoprecipitation (ENP) were tested using three different block copolymers and four incubating water fractions (φ_{w,ENP}). The reconstitution ability after excipient-free lyophilization was assessed through various techniques, including DLS hydrodynamic sizes, TEM images of the reconstituted micelles, and Langmuir force balance analysis (surface pressure-area isotherms) of both the solid lyophilizates and reconstituted micelles. It was found that a faster cooling rate during the freezing stage was advantageous, as it reduced the freezing stress by forming smaller ice crystallites.

Among the variations in block copolymer chemistry, the PEG block end group was identified as a significant factor. The presence of a hydrophobic methoxy end group promoted irreversible aggregation of the micelles. In the case of PS-PEG-OH with a hydroxy end group, a decrease in the size increase factor (SIF) and an increase in surface availability (SA) with higher PEG grafting density were observed. This can be attributed to a higher fraction of hydrated PEG in the micelle formulation, as evidenced by the specific heat of melting at the eutectic point (Tₑᵤ). In the most successful lyophilization process, which involved PS-PEG-OH formulated at φ_{w,ENP}=0.5, the reconstituted suspension exhibited an SA of 87%, and the solid lyophilizate perfectly reproduced the isotherm of the pristine suspension. Based on these results, it is proposed that the amorphous mixture of PEG/water surrounding the micellar core plays a crucial role in dissipating the freezing stress, thereby preventing irreversible aggregation of the micelles.

The above results suggest that BCP micelle PLS can even be delivered as a dry powder aerosol for treatment of lung disorders caused by lung surfactant dysfunction or deficiency, including ARDS, ALI, NRDS, etc. Aerosolized PLS in the form of dry powder or liquid droplets can be delivered to the lungs with the aid of a nebulizer, an inhaler or a mechanical ventilator.

The following numbered statements are provided to further describe the scope of the disclosure:
1. A composition comprising:
solid amphiphilic block copolymer micelles having a hydrodynamic diameter in the range of about 5 nm to about 1,000 nm, the micelles comprising substantially no excipients.
2. The composition of Statement 1, wherein the amphiphilic block copolymer comprises a poly(styrene) and a poly(ethylene glycol) block.
3. The composition of Statement 1, wherein the amphiphilic block copolymer comprises substantially the same number-average molecular weights of PS and PEG blocks.
4. The composition of Statement 1, wherein the PEG-end group of PS-PEG block copolymer is a hydroxyl group.
5. The composition of Statement 1, wherein the micelles have high PEG grafting density σPEG.
6. The composition of Statement 5, wherein the σPEG is about 8 or greater.
7. A kit comprising the composition of Statement 1 in a first container.
8. The kit of Statement 7, wherein the kit further comprises water in a second container.
9. A method of treating a pulmonary disorder in a subject, the method comprising administering to said mammal a therapeutically effective amount of the composition of Statement 1 reconstituted in water.
10. The method of Statement 9, wherein the pulmonary disorder is caused by at least one of a deficiency and the deactivation of functional lung surfactant in the subject.
11. The method of Statement 9, wherein the subject is a mammal.
12. The method of Statement 9, wherein the subject is a human.
13. A method comprising:
(a) dissolving amphiphilic block copolymer in a mixed water-acetone cosolvent;
(b) conducting a single-step dialysis against water to form kinetically frozen monodisperse micelle system in aqueous conditions to give polymer lung surfactant aqueous solution; and
(c) lyophilizing the polymer lung surfactant aqueous solution to give polymer lung surfactant micelles;
wherein the polymer lung surfactant aqueous solution comprises substantially no excipients before the lyophilization.
14. The method of Statement 13, wherein the amphiphilic block copolymer comprises a poly(styrene) and a poly(ethylene glycol) block.
15. The method of Statement 13, wherein the amphiphilic block copolymer comprises substantially the same number-average molecular weights of PS and PEG blocks.
16. The method of Statement 13, wherein the PEG-end group of PS-PEG block copolymer is a hydroxyl group.
17. The method of Statement 13, wherein the step of dissolving comprises:
(a) dissolving PS-PEG block copolymers in a mixed cosolvent with initial acetone composition between 50% and 60% (v/v);
(b) sonicating the solution;
(c) mechanically agitating the solution;
(d) heating the solution; and
(e) leaving the solution quiescent overnight for the equilibration.
18. The method of Statement 13, wherein the step of single-step dialysis comprises:
(a) dialyzing the solution using a dialysis bag against the water reservoir overnight; and
(b) replacing the aqueous reservoir with clean water for at least three times.
19. The method of Statement 13, wherein the lyophilizing comprises:
(a) freezing the aqueous polymer lung surfactant solution;
(b) sublimating crystalized water from the polymer lung surfactant solution until no further water sublimation is detected; and
(c) desorbing and vaporizing the supercooled water adsorbed onto the polymer lung surfactant nanoparticles.
20. The method of Statement 19, wherein the freezing rate is-2 °C/min.
21. The method of Statement 19, wherein the rate of temperature increase between the freezing and the primary drying steps is 1 °C/min.
22. The method of Statement 19, wherein the rate of temperature increase between the primary drying and the secondary drying steps is 1 °C/min.
23. The method of Statement 19, wherein the lyophilized polymer lung surfactant can be potentially used as the aerosolized therapy to treat acute respiratory distress syndrome.
24. A method of reconstituting lyophilized polymer lung surfactant, the method comprising:
(a) dissolving lyophilized polymer lung surfactant of Statement 1 in water;
(b) shaking the dissolved polymer lung surfactant; and
(c) sonicating the dissolved polymer lung surfactant.

1. A composition comprising:
dried amphiphilic block copolymer micelles having a mean hydrodynamic diameter in the range of about 5 nm to 1,000 nm in the original hydrated state, wherein the original micelle structure in an aqueous medium is preserved without aggregation or transformation during the drying process, wherein the drying is done substantially without using any excipients, wherein the original aqueous amphiphilic block copolymer micelle solution is prepared by initially forming amphiphilic block copolymer micelles in a medium containing an organic cosolvent and subsequently dialyzing the micelle solution against an aqueous reservoir.

2. The composition of claim 1, wherein the amphiphilic block copolymer comprises a hydrophobic block comprising a monomer selected from the group consisting of lactic acid or lactide (LA), glycolic acid or glycolide (GA), caprolactone (CL), styrene(S), methacrylate (MA), acrylate (Ac), butadiene (BD), and isoprene (IP), and a hydrophilic block comprising a monomer selected from the group consisting of ethylene glycol (EG), ethylene oxide (EO), vinyl alcohol (VA), oxazoline (OZ), and acrylamide (AM).

3. (canceled)

4. The composition of claim 1, wherein the amphiphilic block copolymer comprises a poly(styrene) (PS) block having a number-average molecular weight in the range of 0.5-500 kDa and a poly(ethylene glycol) (PEG) block having a number-average molecular weight in the range of 0.5-500 kDa.

5. The composition of claim 1, wherein the corona-forming hydrophilic block of the amphiphilic block copolymer has a hydrophilic end group.

6. (canceled)

7. The composition of claim 1, wherein the micelles have a high dimensionless corona chain grafting density σ_{corona}, wherein σ_{corona} is defined as the projected area of the hydrophilic block segment in its unperturbed state divided by the micelle core surface area per hydrophilic segment.

8. The composition of claim 7, wherein the σ_{corona} is about 5 or greater.

9. A kit comprising the composition of claim 1 in a first container and optionally, the kit further comprises an aqueous liquid in a second container.

10. (canceled)

11. A method of treating a pulmonary disorder in a subject, the method comprising administering to said mammal a therapeutically effective amount of the composition of claim 1 in the dried state or after reconstitution in an aqueous medium.

12. The method of claim 11, wherein the pulmonary disorder is caused by deficiency and/or deactivation of endogenous lung surfactant in the subject.

13. (canceled)

14. (canceled)

15. A method comprising:
(a) dissolving an amphiphilic block copolymer in a mixed water-organic cosolvent mixture;
(b) conducting a single-step dialysis against an aqueous reservoir to form a kinetically frozen monodisperse micelle polymer lung surfactant system in an aqueous solution; and
(c) drying the aqueous polymer lung surfactant micelle solution to produce dried polymer lung surfactant micelles;
wherein the aqueous polymer lung surfactant solution comprises substantially no excipients before the drying process.

16. The method of claim 15, wherein the amphiphilic block copolymer comprises a hydrophobic block comprising a monomer selected from the group consisting of lactic acid or lactide (LA), glycolic acid or glycolide (GA), caprolactone (CL), styrene(S), methacrylate (MA), acrylate (Ac), butadiene (BD), and isoprene (IP), and a hydrophilic block comprising a monomer selected from the group consisting of ethylene glycol (EG), ethylene oxide (EO), vinyl alcohol (VA), oxazoline (OZ), and acrylamide (AM).

17. (canceled)

18. The method of claim 15, wherein the amphiphilic block copolymer comprises a PS block having a number-average molecular weight in the range of 0.5-500 kDa and a PEG block having a number-average molecular weight in the range of 0.5-500 kDa.

19. The method of claim 15, wherein the corona-forming hydrophilic block of the amphiphilic block copolymer has a hydrophilic end group.

20. (canceled)

21. The method of claim 15, wherein the step of dissolving comprises:
(a) dissolving block copolymers in a water-organic cosolvent mixture at an initial water composition between about 10 and 90% (v/v); and
(b) sonicating or mechanically agitating or heating the solution;
(c) keeping the solution quiescently or under mild stirring for at least about ten minutes for equilibration.

22. The method of claim 15, wherein the step of single-step dialysis comprises:
(a) dialyzing the solution using a dialysis bag against an aqueous reservoir for at least about ten minutes; and
(b) replacing the aqueous reservoir with a fresh aqueous medium at least once during the dialysis period.

23. The method of claim 15, wherein the drying process comprises:
(a) freezing the aqueous polymer lung surfactant micelle solution;
(b) sublimating crystalized water from the polymer lung surfactant micelle solution until no further water sublimation is detected; and
(c) desorbing and vaporizing the supercooled water adsorbed to the polymer lung surfactant nanoparticles (micelles).

24. The method of claim 23, wherein the freezing rate is about −2 °C/min.

25. The method of claim 23, wherein the rate of temperature increase between the freezing and the primary drying steps is about 1 °C/min or the rate of temperature increase between the primary drying and the secondary drying steps is about 1 °C/min.

26. (canceled)

27. The method of claim 23, wherein the dried polymer lung surfactant micelles can be used as a therapy in dry powder form or after reconstitution in an aqueous medium to treat acute respiratory distress syndrome, acute lung injury or neonatal respiratory distress syndrome.

28. (canceled)

29. A method of reconstituting dried polymer lung surfactant micelles, the method comprising:
(a) dissolving the dried polymer lung surfactant micelles of claim 1 in an aqueous medium; and
(b) shaking or vortexing or mechanically agitating or sonicating the dissolved polymer lung surfactant micelles.
